(19) United States
(12) Patent Application Publication
(10) Pub. No.: US 2023/0165653 A1
(43) Pub. Date: Jun. 1, 2023

(54) SYSTEMS, METHODS, AND DEVICES FOR COVERING AND TRACKING A SURGICAL DEVICE

(71) Applicant: Mazor Robotics Ltd., Caesarea (IL)

(72) Inventors: Ziv Seemann, Beit Ytzhack (IL); Adi Sandelson, Givatayim (IL); Bat-el Kagan, Natanya (IL); Dvir Kadshai, Tel Aviv (IL); Yuval A. Chen, Tel Aviv-Yafo (IL); Yvan R. Paitel, Louisville, CO (US); Nicholas J. Rawluk, Boulder, CO (US)

(21) Appl. No.: 17/536,422

(22) Filed: Nov. 29, 2021

(51) Int. Cl. A61B 46/10; A61B 34/20; A61B 90/00; A61B 90/96; A61B 34/30

(52) U.S. Cl. CPC A61B 46/10; A61B 34/20; A61B 90/39; A61B 90/96; A61B 34/30

(57) ABSTRACT

Systems, methods, and devices for covering and tracking a surgical device are provided. The system may comprise a flexible sheet configured to cover a surgical device, at least one marker disposed on a first side of the flexible sheet, a connector disposed on a second side of the flexible sheet, a receiver attached to a surgical device and configured to receive the connector. The receiver may be configured to align and hold the flexible sheet to the surgical device.

### BACKGROUND

The present disclosure is generally directed to coverings, and relates more particularly to coverings comprising a flexible sheet having a marker for tracking and a connector for connecting the flexible sheet to a surgical device.

Surgical robots may assist a surgeon or other medical provider in carrying out a surgical procedure, or may complete one or more surgical procedures autonomously in a sterile environment. Providing controllable linked articulating members allows a surgical robot to reach areas of a patient anatomy during various medical procedures.

### BRIEF SUMMARY

Example aspects of the present disclosure include:

A system for covering and tracking a surgical device according to at least one embodiment of the present disclosure comprises a cover assembly comprising: a flexible sheet configured to cover a surgical device; at least one marker disposed on a first side of the flexible sheet; a connector disposed on a second side of the flexible sheet, the second side opposite the first side; and a receiver attached to a surgical device and configured to receive the connector, the receiver configured to align and hold the flexible sheet to the surgical device.

Any of the aspects herein, wherein the at least one marker is preconfigured on the flexible sheet prior to installation of the flexible sheet on the surgical device.

Any of the aspects herein, wherein at least one of the receiver or the connector comprises a magnet and at least another one of the receiver or the connector comprises steel.

Any of the aspects herein, wherein the surgical device comprises a robotic arm and the at least one marker corresponds to a face of a plurality of faces of the robotic arm.

Any of the aspects herein, further comprising: a processor; and a memory storing data for processing by the processor, the data, when processed, causing the processor to: receive information about the at least one marker; and identify a face of the robotic arm based on the information.

Any of the aspects herein, wherein the memory store further data for processing by the processor that, when processed, causes the processor to: track the face.

Any of the aspects herein, wherein the information comprises a pattern formed by the at least one marker, the pattern corresponding to the face of the robotic arm.

Any of the aspects herein, wherein the information comprises a pose of the at least one marker, and wherein the memory store further data for processing by the processor that, when processed, causes the processor to: receive a pose of the robotic arm, and wherein identifying the face of the robotic arm is based on the pose of the robotic arm and the pose of the at least one marker.

Any of the aspects herein, wherein the receiver and the connector form a kinematic coupling.

Any of the aspects herein, wherein the at least one marker comprises at least one of a reflective sticker, a barcode, or a QR code.

Any of the aspects herein, wherein the flexible sheet forms a sterile barrier between the surgical device and a sterile environment.

Any of the aspects herein, wherein the receiver is at least one of integrated with the surgical device or attached to the surgical device.

A system for covering and tracking a surgical device according to at least one embodiment of the present disclosure comprises a flexible sheet configured to cover a surgical device: at least one marker disposed on a first side of the flexible sheet; a connector disposed on a second side of the flexible sheet, the second side opposite the first side; a robotic arm having a receiver configured to receive the connector, the receiver configured to align and hold the flexible sheet to the surgical device; a processor; and a memory storing data for processing by the processor, the data, when processed, causing the processor to: receive information about the at least one marker; identify a face of the robotic arm based on the information; and track the face.

Any of the aspects herein, wherein the information comprises a pose of the at least one marker, and wherein the memory store further data for processing by the processor that, when processed, causes the processor to: receive a pose of the robotic arm, and wherein identifying the face of the robotic arm is based on the pose of the robotic arm and the pose of the at least one marker.

Any of the aspects herein, wherein the receiver and the connector form a kinematic coupling.

Any of the aspects herein, wherein the information comprises a pattern formed by the at least one marker, the pattern corresponding to the face of the robotic arm.

Any of the aspects herein, wherein the at least one marker is preconfigured on the flexible sheet prior to installation of the flexible sheet on the surgical device.

Any of the aspects herein, wherein at least one of the receiver or the connector comprises a magnet and at least another one of the receiver or the connector comprises steel.

Any of the aspects herein, wherein the at least one marker comprises at least one of a reflective sticker, a barcode, or a QR code.

A device for covering and tracking a surgical device according to at least one embodiment of the present disclosure comprises a processor; and a memory storing data for processing by the processor, the data, when processed, causing the processor to: receive information about at least one marker disposed on a first side of a flexible sheet, the flexible sheet configured to cover and couple to a robotic arm via a connector disposed on a second side of the flexible sheet and a receiver disposed on the robotic arm; identify a face of a robotic arm based on the information; and track the face.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a schematic illustration of a first view of a cover assembly according to at least one embodiment of the present disclosure;

FIG. 1B is a schematic illustration of a second view of a cover assembly according to at least one embodiment of the present disclosure;

FIG. 2 is a schematic illustration of a cover assembly and a robotic arm according to at least one embodiment of the present disclosure;

FIG. 3 is a block diagram of a system according to at least one embodiment of the present disclosure; and

FIG. 4 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Convention navigation trackers may include sterile trackers that are attached to a component; one-time use trackers such as spheres or a reflective lens sold under the trademark RADIX™; autoclavable trackers that incorporate infrared light emitting diodes (IRED); and/or non-autoclavable trackers that incorporate IREDs that go through a drape. During a surgical operation, a surgical device such as a robot may be draped and trackers may be attached to the robot after the robot is draped (e.g., robotic reference frame, cranial frame). Such procedures take time and pierces the drape during the process. As a result, removing a tracker due to failure of the item is not possible. Further, the drape quality may affect and reduce an accuracy and quality of navigation if the drape obscures trackers positioned under the drape. Additionally, if multiple trackers are placed on a surgical device such as a robot, time in the operating room is increased to allow for draping and to place the trackers accurately.

In at least one embodiment of the present disclosure, markers such as, for example, reflective stickers may be attached to a drape or flexible sheet, and thus eliminate the alignment of the robot trackers and the drape straightening process. The robot may comprise a kinematic coupling for receiving the markers, which will keep the marker and the drape or flexible sheet in place (e.g., a magnet on a robot and steel on the drape). During placement of the drape or flexible sheet, the drape or flexible sheet may include information such as, for example, a barcode to scan, which may give the location of the markers and the corresponding kinematic coupling. Such information will help a user to drape the robot (or other device) quickly and with ease. This advantageously reduces time in the operating room for autoclavable items (e.g., tracker base for sphere and sphere placement time on other components). With this solution the operating room can be occupied for less time, have better accuracy for navigation, decreased risk due to drape tear, and/or an option for cost reduction due to the use of cheaper stickers.

Embodiments of the present disclosure provide technical solutions to one or more of the problems of (1) simultaneously placing a marker on a surgical device and covering the surgical device with a flexible sheet, (2) increasing an accuracy of tracking one or more faces of a surgical device, (3) providing a marker preconfigured on a flexible sheet; and (4) decreasing an amount of time to prepare a surgical device for a surgical operation.

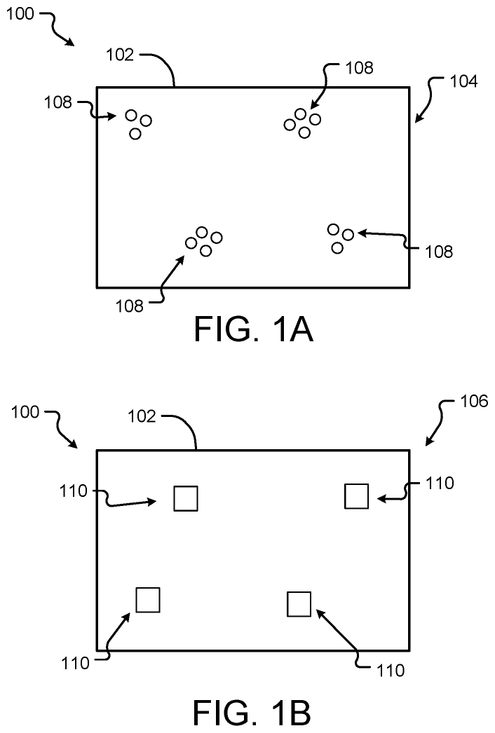

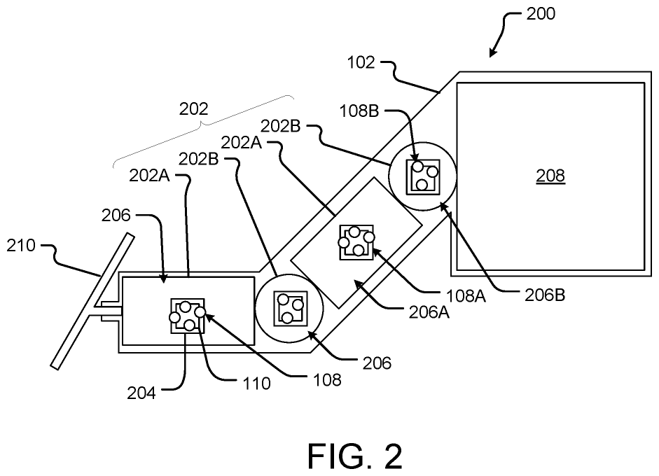

Turning first to FIGS. 1A and 1B, a first view of a cover assembly 100 and a second view of the cover assembly 100 are shown respectively. The cover assembly 100 is configured to cover a surgical device 200 (shown in FIG. 2) and provide tracking of one or more elements, objects, and/or faces of the surgical device 200. The cover assembly 100 comprises a flexible sheet 102 having a first side 104 (shown in FIG. 1A) and a second side 106 (shown in FIG. 1B). The flexible sheet 102 is configured to cover a surgical device 200 (shown and described in FIG. 2). The surgical device 200 may be, for example, a robotic arm such as a robotic arm 202 (also shown and described in FIG. 2). In some embodiments, the flexible sheet 102 may be sterile or may be sterilized, and may form a sterile boundary between the surgical device 200 and a sterile environment. For example, a sterile boundary may be formed between a robotic arm (which may not be sterile) and a surgical site such as, for example, a spinal region of a patient. The flexible sheet 102 may be transparent, opaque, or semi-transparent. In some embodiments, the flexible sheet 102 may comprise a combination of transparent, opaque, and/or semi-transparent portions. In the illustrated embodiment, the flexible sheet 102 is shown as a rectangular sheet for illustrative purposes. It will be appreciated that in other embodiments, the flexible sheet 102 may be any shape or size. The flexible sheet 102, in some embodiments, may be contoured to precisely fit over the surgical device. The flexible sheet 102 may also be shaped to allow movement of the surgical device. For example, the flexible sheet 102 may comprise an articulated area positioned at, for example, a joint of a robotic arm to enable articulated movement of the joint.

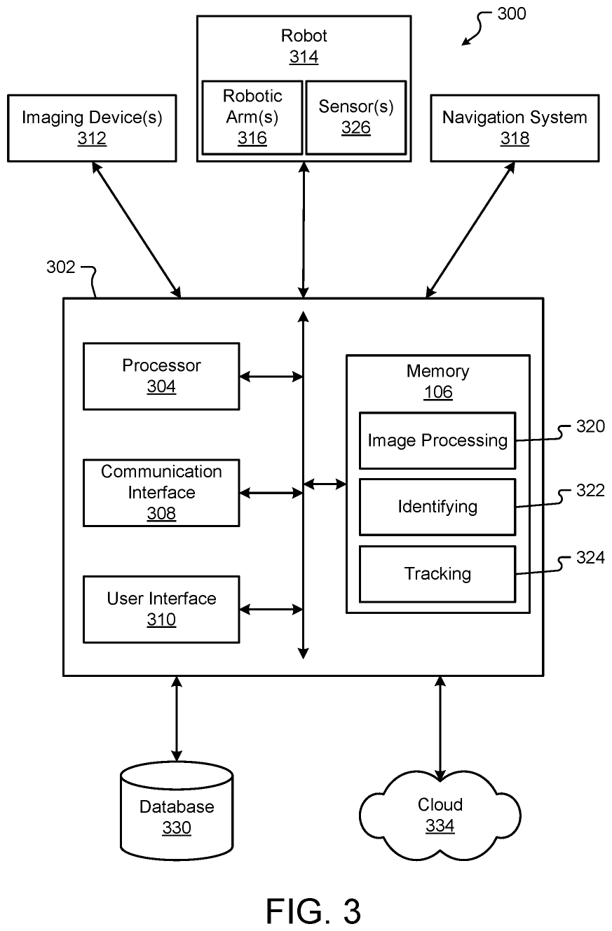

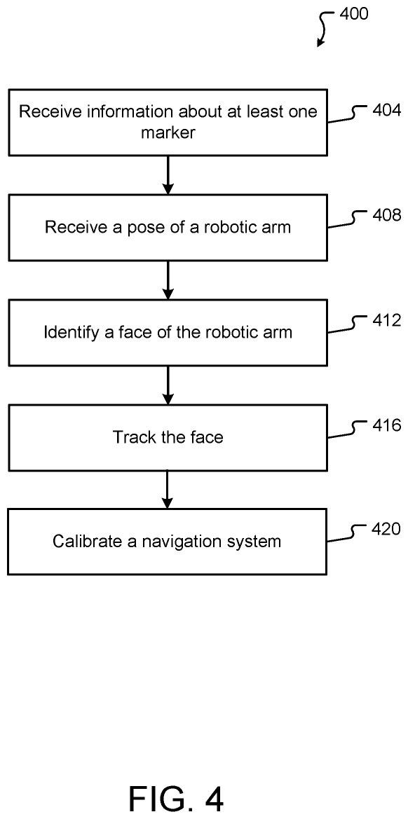

The cover assembly 100 also comprises at least one marker 108 disposed on the first side 104 of the flexible sheet 102. The marker 108 may comprise a passive marker. For example, the marker 108 may comprise, for example, reflective stickers, spheres, QR codes, barcodes, printed pattern(s), or any combination thereof. In other embodiments, the marker 108 may comprise an active marker or a combination of passive and active markers. In some embodiments, the marker 108 may be adhered, stitched, or otherwise attached to the flexible sheet 102. In other embodiments, the marker 108 may be printed, painted, deposited, or sprayed onto the flexible sheet 102. The markers 108 may be used to track the surgical device 200. More specifically, in some embodiments wherein the surgical device 200 is a robotic arm 202, the markers 108 may be used to track the robotic arm 202 (using, for example, a navigation system such as a navigation system 318, shown and described in FIG. 3). In some instances, the markers 108 may be used to track a plurality of faces 206 on the robotic arm 202, as will be described in detail in FIGS. 2 and 4. In the illustrated embodiment, the at least one marker 108 forms four sets of markers for tracking a corresponding four faces 206. It will be appreciated that in other embodiments, the at least one marker 108 may form less than or more than four sets of markers to track four faces, less than four faces, or more than four faces of the surgical device 200.

The cover assembly 100 also comprises at least one connector 110 disposed on the second side 106 of the flexible sheet 102. In the illustrated embodiment, the at least one connector 110 comprises four connectors, though it will be appreciated that in other embodiments the at least one connector 110 may comprise less than or more than four connectors. The connector 110 is configured to align and hold the flexible sheet 102 to the surgical device. The connector 110 is receivable by a receiver 204 (shown in FIG. 2) of the surgical device 200. The connector 110 and the receiver 204 may together form a kinematic coupling. For example, at least one of the connector 110 or the receiver 204 may comprise a plurality of spheres and another one of the connector 110 or the receiver 204 may comprise a plurality of grooves configured to receive the plurality of spheres. In other embodiments, at least one of the connector 110 or the receiver 204 may comprise a magnet and another one of the connector 110 or the receiver 204 may comprise a metal configured to mate with the magnet.

In some embodiments, the connector 110 couples to a corresponding receiver 204 so as to position the flexible sheet 102 in a predetermined configuration over the surgical device 200. The configuration may enable the positioning of the markers 108 in specific positions on the surgical device 200. In particular, the markers 108 may be positioned such that a plurality of faces 206 (shown in FIG. 2) of the surgical device 200 can be identified by the corresponding markers 108. For example, a first set of markers 108A may be positioned on a first face 206A and a second set of markers 108B may be positioned on a second face 206B. The first set of markers 108A and the second set of markers 108B may be unique to each respective face 206A, 206B. In other words, the first set of markers 108 may be, for example, a set of stickers disposed in a first pattern and the second set of markers 108 may be a set of stickers disposed in a second pattern. In other instances, the first set of markers 108 and the second set of markers 108 may be the same and the corresponding face 206 of the surgical device 200 may be determined using additional information (e.g., a pose of surgical device 200, as will be discussed in detail with respect to FIG. 4).

In some embodiments, the marker 108 and the connector 110 may be integrated. For example, the connector 110 may comprise a screw and the marker 108 may be disposed on an end of the screw. In the same embodiments, the receiver 204 may comprise a threaded bore formed on the surgical device 200. The screw is then screwed through the flexible sheet 102 and into the threaded bore to couple the flexible sheet 102 to the surgical device 200.

It will be appreciated that although the connectors 110 are not visible in FIG. 1A and the markers 108 are not visible in FIG. 1B, that in embodiments where the flexible sheet 102 is transparent, the connectors 110 and/or the markers 108 may be visible through the flexible sheet 102 on either side of the flexible sheet 102.

Turning to FIG. 2, the cover assembly 100 is shown covering the surgical device 200. As previously described, the cover assembly 100 enables tracking of the one or more faces without interference from the flexible sheet 102 (e.g., wrinkles obscuring markers under the drape). The cover assembly 100 enables simultaneous installation of markers 108 and the flexible sheet 108, thereby reducing an amount of time to prepare the surgical device 200 for, for example, a surgical operation.

The surgical device 200, in the illustrated embodiment, is a robotic arm 202 comprising one or more members 202A connected by one or more joints 202B extending from a base 208 that may be stationary or movable. It will be appreciated that in other embodiments, the surgical device 200 may be any device such as, for example, an imaging device, a stand, a monitor, or any component that may be useful during a surgical operation. Though the base 208 is covered by the flexible sheet 102 in the illustrated embodiment, it will be appreciated that the base 208 may not be covered by the flexible sheet 102 in other embodiments. An end effector 210 may be disposed at an end of the robotic arm 202. Though the end effector 210 is not covered by the flexible sheet 102 in the illustrated embodiment, it will be appreciated that the end effector 210 may be covered by the flexible sheet 102 in other embodiments.

The robotic arm 202 comprises a plurality of faces 206 (which may also be referred to as surfaces) and at least one receiver 204 corresponding to the at least one connector 110. The at least one receiver 204 may be integrated with the robotic arm 202 or may be affixed to the robotic arm 202. As previously described, the receiver 204 and the connector 110 may form a kinematic coupling. The receiver 204 may comprise, for example, a magnet, a metal, a groove, a hook and loop fabric, or combinations thereof. The receiver 204 may be permanently affixed or integrated with the robotic arm 202, or may be removably affixed to the robotic arm 202.

In the illustrated embodiment each of the joints 202B and the members 202A comprise a face 206. A face 206 may be, for example, a flat surface, a textured surface, a curved surface, a surface of any shape, or a combination thereof. The face 206 may also include a number of surfaces (curved or flat) that are adjacent to one another. In some embodiments, the face 206 may comprise surfaces that are movable (whether movable relative to other surfaces of the face 206 or movable relative to other surfaces of an adjacent face 206), surfaces that are stationary, or a combination thereof.

It will be appreciated that in some embodiments, each joint 202B and/or each member 202A may comprise more than one face. In the illustrated embodiment, a corresponding marker 108 is positioned on each face 206. In other embodiments, a marker 108 may not be positioned on each face 206 and only certain faces 206 may include a marker 108. The marker 108 is used to track and/or identify the corresponding face 206 during, for example, a surgical operation. The marker 108 may be tracked by, for example, a navigation system such as the navigation system 318. In some embodiments, the marker 108 may be unique to the face 206, though in other embodiments, each marker 108 may be the same regardless of which face 206 the marker 108 corresponds with. The markers 108 are aligned with each face by way of positioning the flexible sheet 102 on the robotic arm 202 and using the connectors 110 and the receivers 204 to align and hold the flexible sheet 102 on the robotic arm 202.

In some embodiments, instructions for positioning the connector 110 with a corresponding receiver 204 may be provided. Such instructions may be provided with the cover assembly 100, may be accessed online via a user interface such as a user interface 310, or otherwise provided to a user. In some embodiments, a barcode or QR code (or any other identification) may be supplied on the flexible sheet 102, on or with packaging used to transport the cover assembly 100, or otherwise supplied to the user. The barcode or QR code may be scanned, which may then link to or supply instructions for installing the flexible sheet 102 onto the surgical device 200.

It will be appreciated that though the illustrated embodiment shows the markers 108 and the connectors 110 aligned and in the same position with each other on the flexible sheet 102, that in other embodiments, the markers 108 and the connectors 110 may not be aligned with each other, and may each be positioned on different portions of the flexible sheet 102.

In some embodiments, the cover assembly 100 is reusable. In other embodiments, the cover assembly 100 is single-time use and not reusable. Further, some components of the cover assembly 100 may be reusable and other components may be single-time use. For example, the connector 110 may be reusable and the flexible sheet 102 and the marker 108 may not be reusable. In other examples, the connector 110 and the flexible sheet 102 may be reusable and the marker 108 may not be reusable. In another example, the connector 110 and the marker 108 may be reusable and the flexible sheet 102 may not be reusable. In still other examples, the flexible sheet 102 may be reusable and the connector 110 and the marker 108 may not be reusable.

The cover assembly 100 may be shipped fully assembled. In other words, the marker 108 and the connector 110 may be preinstalled or preconfigured on the flexible sheet 102 prior to being transported to, for example, a surgical site. By having the marker 108 (and/or the connector 110) preinstalled on the flexible sheet 102, overall operating time is reduced as the marker 108 does not need to be separately installed onto the surgical device 200. In other words, installation of the flexible sheet 102 onto the surgical device 200 simultaneously installs the marker 108 onto the surgical device 200. In other instances, the cover assembly 100 may be transported to a surgical site partially assembled or as individual components. For example, the connector 110 may be installed on the flexible sheet 102 prior to transportation to a surgical site, then the marker 108 (whether shipped together or separately from the flexible sheet 102 and/or the connector 110) may be installed onto the flexible sheet 102 at the surgical site. In other examples, the flexible sheet 102, the connector 110, and the marker 108 may be transported unassembled.

Turning to FIG. 3, a block diagram of a system 300 according to at least one embodiment of the present disclosure is shown. The system 300 may be used to identify and track a surgical device such as the surgical device 200 and/or one or more faces such as the faces 206 of the surgical device 200 and/or carry out one or more other aspects of one or more of the methods disclosed herein. The system 300 comprises a computing device 302, one or more imaging devices 312, a robot 314, a navigation system 318, a database 330, and/or a cloud or other network 334. Systems according to other embodiments of the present disclosure may comprise more or fewer components than the system 300. For example, the system 300 may not include the imaging device 312, the robot 314, the navigation system 318, one or more components of the computing device 302, the database 330, and/or the cloud 334.

The computing device 302 comprises a processor 304, a memory 306, a communication interface 308, and a user interface 310. Computing devices according to other embodiments of the present disclosure may comprise more or fewer components than the computing device 302.

The processor 304 of the computing device 302 may be any processor described herein or any similar processor. The processor 304 may be configured to execute instructions stored in the memory 306, which instructions may cause the processor 304 to carry out one or more computing steps utilizing or based on data received from the imaging device 312, the robot 314, the navigation system 318, the database 330, and/or the cloud 334.

The memory 306 may be or comprise RAM, DRAM, SDRAM, other solid-state memory, any memory described herein, or any other tangible, non-transitory memory for storing computer-readable data and/or instructions. The memory 306 may store information or data useful for completing, for example, any step of the method 400 described herein, or of any other methods. The memory 306 may store, for example, instructions and/or machine learning models that support one or more functions of the robot 314. For instance, the memory 306 may store content (e.g., instructions and/or machine learning models) that, when executed by the processor 304, enable image processing 320, identifying 322, and/or tracking 324.

The image processing 320 enables the processor 304 to process image data of an image (received from, for example, the imaging device 312, an imaging device of the navigation system 318, or any imaging device) for the purpose of, for example, identifying information about at least one marker such as the markers 108 depicted in the image. The information may comprise, for example, a pose of the markers, a pattern defined by the markers, a QR code, or a barcode. The information obtained from the image processing 320 may enable the navigation system 318 to identify a face 206 of the surgical device 200 corresponding to the marker 108.

The identifying 322 enables the processor 304 to identify the face 206 of the surgical device 200 corresponding to the marker 108. The identifying 322 may, for example, enable the processor 304 to match a unique identifier such as a unique pattern formed by the marker 108 (whether the unique pattern is, for example, a combination of stickers in a unique pattern, a barcode, or a QR code) with a corresponding face. The unique pattern and the corresponding face may be predetermined and stored in, for example, the memory 306, the database 330, or the cloud 334 and may accessed by the processor 304 when executing the identifying 322. In other embodiments, the identifying 322 may, for example, enable the processor 304 use pose information of the surgical device 200 (which may be received from the surgical device 200 or otherwise) and identify the face 206 corresponding to the marker 108 based on the pose of the surgical device 200 and the information about the marker 108 obtained from the image processing 320.

The tracking 324 enables the processor 304 to track the face 206 identified by the identifying 222. The tracking 324 may, for example, enable the processor 304 to compare the identified face 206 at a first time period and a second time period to determine if movement of the identified face 206 has occurred. In other embodiments, the tracking 324 may, for example, enable the processor 304 to compare a pose of the identified face 206 (whether determined from image processing 320, receiving a pose from the surgical device 200, or otherwise) at a first time period and a second time period to determine a change in the pose (and thus, movement of the identified face 206).

The content stored in the memory 306, if provided as in instruction, may, in some embodiments, be organized into one or more applications, modules, packages, layers, or engines. Alternatively or additionally, the memory 306 may store other types of content or data (e.g., machine learning models, artificial neural networks, deep neural networks, etc.) that can be processed by the processor 304 to carry out the various method and features described herein. Thus, although various contents of memory 306 may be described as instructions, it should be appreciated that functionality described herein can be achieved through use of instructions, algorithms, and/or machine learning models. The data, algorithms, and/or instructions may cause the processor 304 to manipulate data stored in the memory 306 and/or received from or via the imaging device 312, the robot 314, the database 330, and/or the cloud 334.

The computing device 302 may also comprise a communication interface 308. The communication interface 308 may be used for receiving image data or other information from an external source (such as the imaging device 312, the robot 314, the navigation system 318, the database 330, the cloud 334, and/or any other system or component not part of the system 300), and/or for transmitting instructions, images, or other information to an external system or device (e.g., another computing device 302, the imaging device 312, the robot 314, the navigation system 318, the database 330, the cloud 334, and/or any other system or component not part of the system 300). The communication interface 308 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 308 may be useful for enabling the device 302 to communicate with one or more other processors 304 or computing devices 302, whether to reduce the time needed to accomplish a computing-intensive task or for any other reason.

The computing device 302 may also comprise one or more user interfaces 310. The user interface 310 may be or comprise a keyboard, mouse, trackball, monitor, television, screen, touchscreen, and/or any other device for receiving information from a user and/or for providing information to a user. The user interface 310 may be used, for example, to receive a user selection or other user input regarding any step of any method described herein. Notwithstanding the foregoing, any required input for any step of any method described herein may be generated automatically by the system 300 (e.g., by the processor 304 or another component of the system 300) or received by the system 300 from a source external to the system 300. In some embodiments, the user interface 310 may be useful to allow a surgeon or other user to modify instructions to be executed by the processor 304 according to one or more embodiments of the present disclosure, and/or to modify or adjust a setting of other information displayed on the user interface 310 or corresponding thereto.

Although the user interface 310 is shown as part of the computing device 302, in some embodiments, the computing device 302 may utilize a user interface 310 that is housed separately from one or more remaining components of the computing device 302. In some embodiments, the user interface 310 may be located proximate one or more other components of the computing device 302, while in other embodiments, the user interface 310 may be located remotely from one or more other components of the computer device 302.

The imaging device 312 may be operable to image markers such as markers 108, anatomical feature(s) (e.g., a bone, veins, tissue, etc.), and/or other aspects of patient anatomy to yield image data (e.g., image data depicting or corresponding to a marker, bone, veins, tissue, etc.). “Image data” as used herein refers to the data generated or captured by an imaging device 312, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 312 may be used to obtain first image data (e.g., a first image) at a first time, and a second imaging device 312 may be used to obtain second image data (e.g., a second image) at a second time after the first time. The imaging device 312 may be capable of taking a 2D image or a 3D image to yield the image data.

The imaging device 312 may be or comprise, for example, an optical camera, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 312 suitable for obtaining images of a marker or an anatomical feature of a patient. The imaging device 312 may be contained entirely within a single housing, or may comprise a transmitter/emitter and a receiver/detector that are in separate housings or are otherwise physically separated.

In some embodiments, the imaging device 312 may comprise more than one imaging device 312. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 312 may be operable to generate a stream of image data. For example, the imaging device 312 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 314 may be any surgical robot or surgical robotic system. The robot 314 may be or comprise, for example, the Mazor X™ Stealth Edition robotic guidance system. The robot 314 may be configured to position the imaging device 312 at one or more precise position(s) and orientation(s), and/or to return the imaging device 312 to the same position(s) and orientation(s) at a later point in time. The robot 314 may additionally or alternatively be configured to manipulate a surgical tool (whether based on guidance from the navigation system 318 or not) to accomplish or to assist with a surgical task. In some embodiments, the robot 314 may be configured to hold and/or manipulate an anatomical element during or in connection with a surgical procedure. The robot 314 may comprise one or more robotic arms 316 which may be the same as or similar to the robotic arm 202. In some embodiments, the robotic arm 316 may comprise a first robotic arm and a second robotic arm, though the robot 314 may comprise more than two robotic arms. In some embodiments, one or more of the robotic arms 316 may be used to hold and/or maneuver the imaging device 312. In embodiments where the imaging device 312 comprises two or more physically separate components (e.g., a transmitter and receiver), one robotic arm 316 may hold one such component, and another robotic arm 316 may hold another such component. Each robotic arm 316 may be positionable independently of the other robotic arm. The robotic arms 316 may be controlled in a single, shared coordinate space, or in separate coordinate spaces.

The robot 314, together with the robotic arm 316, may have, for example, one, two, three, four, five, six, seven, or more degrees of freedom. Further, the robotic arm 316 may be positioned or positionable in any pose, plane, and/or focal point. The pose includes a position and an orientation. As a result, an imaging device 312, surgical tool, or other object held by the robot 314 (or, more specifically, by the robotic arm 316) may be precisely positionable in one or more needed and specific positions and orientations.

The robotic arm(s) 316 may comprise one or more sensors 326 that enable the processor 304 (or a processor of the robot 314) to determine a precise pose in space of the robotic arm 316 (as well as any object or element held by or secured to the robotic arm). The sensors 326 may positioned adjacent to or integrated with the robotic arm 316. The one or more sensors 326 may include a plurality of sensors and each sensor 326 may be positioned at the same location or a different location as any other sensor 326.

The sensors 326 may correspond to transducers that are configured to convert physical phenomena into an electrical signal that is capable of being processed by the processor 304. The sensor 326 may include one or more or any combination of components that are electrical, mechanical, electro-mechanical, magnetic, electromagnetic, or the like. Non-limiting examples of sensors 326 include gyroscopic sensors, accelerometers, strain gauges, impact sensors, vibration detectors, a torque sensor, a force sensor, a linear encoder, a rotary encoder, a capacitor, and/or an accelerometer. In some embodiments, the sensor 326 may include a memory for storing sensor data. In still other examples, the sensor 326 may output signals (e.g., sensor data) to one or more sources (e.g., the computing device 302 and/or the navigation system 318).

In some embodiments, markers (e.g., navigation markers) may be placed on the robot 314 (including, e.g., on the robotic arm 316) via the covering assembly 100. In other embodiments, markers may also be placed on the imaging device 312 or any other object in the surgical space. The markers (which may be the same as or similar to the markers 108 and/or may also be references markers separate from the markers 108) may be tracked by the navigation system 318, and the results of the tracking may be used by the robot 314 and/or by an operator of the system 300 or any component thereof. In some embodiments, the navigation system 318 can be used to track other components of the system (e.g., imaging device 312) and the system can operate without the use of the robot 314 (e.g., with the surgeon manually manipulating the imaging device 312 and/or one or more surgical tools, based on information and/or instructions generated by the navigation system 318, for example).

The navigation system 318 may provide navigation for a surgeon and/or a surgical robot during an operation. The navigation system 318 may be any now-known or future-developed navigation system, including, for example, the Medtronic StealthStation™ S8 surgical navigation system or any successor thereof. The navigation system 318 may include one or more cameras or other sensor(s) for tracking at least one marker 108, navigated trackers, or other objects within the operating room or other room in which some or all of the system 300 is located. The one or more cameras may be optical cameras, infrared cameras, or other cameras. In some embodiments, the navigation system 318 may comprise one or more electromagnetic sensors. In various embodiments, the navigation system 318 may be used to track a position and orientation (e.g., a pose) of the imaging device 312, the robot 314 and/or robotic arm 316, and/or one or more surgical tools (or, more particularly, to track a pose of a navigated tracker attached, directly or indirectly, in fixed relation to the one or more of the foregoing). The navigation system 318 may include a display for displaying one or more images from an external source (e.g., the computing device 302, imaging device 312, or other source) or for displaying an image and/or video stream from the one or more cameras or other sensors of the navigation system 318. In some embodiments, the system 300 can operate without the use of the navigation system 318. The navigation system 318 may be configured to provide guidance to a surgeon or other user of the system 300 or a component thereof, to the robot 314, or to any other element of the system 300 regarding, for example, a pose of one or more anatomical elements, whether or not a tool is in the proper trajectory, and/or how to move a tool into the proper trajectory to carry out a surgical task according to a preoperative or other surgical plan.

The database 330 may store information that correlates one coordinate system to another (e.g., one or more robotic coordinate systems to a patient coordinate system and/or to a navigation coordinate system). The database 330 may additionally or alternatively store, for example, corresponding markers 108 and faces 206 of the surgical device 200; one or more surgical plans (including, for example, pose information about a target and/or image information about a patient's anatomy at and/or proximate the surgical site, for use by the robot 314, the navigation system 318, and/or a user of the computing device 302 or of the system 300); one or more images useful in connection with a surgery to be completed by or with the assistance of one or more other components of the system 300; and/or any other useful information. The database 330 may be configured to provide any such information to the computing device 302 or to any other device of the system 300 or external to the system 300, whether directly or via the cloud 334. In some embodiments, the database 330 may be or comprise part of a hospital image storage system, such as a picture archiving and communication system (PACS), a health information system (HIS), and/or another system for collecting, storing, managing, and/or transmitting electronic medical records including image data.

The cloud 334 may be or represent the Internet or any other wide area network. The computing device 302 may be connected to the cloud 334 via the communication interface 308, using a wired connection, a wireless connection, or both. In some embodiments, the computing device 302 may communicate with the database 330 and/or an external device (e.g., a computing device) via the cloud 334.

The system 300 or similar systems may be used, for example, to carry out one or more aspects of any of the method 400 described herein. The system 300 or similar systems may also be used for other purposes.

FIG. 4 depicts a method 400 that may be used, for example, identifying and tracking a surgical device.

The method 400 (and/or one or more steps thereof) may be carried out or otherwise performed, for example, by at least one processor. The at least one processor may be the same as or similar to the processor(s) 304 of the computing device 302 described above. The at least one processor may be part of a robot (such as a robot 314) or part of a navigation system (such as a navigation system 318). A processor other than any processor described herein may also be used to execute the method 400. The at least one processor may perform the method 400 by executing elements stored in a memory such as the memory 306. The elements stored in memory and executed by the processor may cause the processor to execute one or more steps of a function as shown in method 400. One or more portions of a method 400 may be performed by the processor executing any of the contents of memory, such as an image processing 320, identifying 322, and/or a tracking 324.

The method 400 comprises receiving information about at least one marker (step 404). The at least one marker may be the same as or similar to the at least one marker 108. The at least one marker may be attached to or integrated with a first side of a flexible sheet such as the flexible sheet 102, which may be configured to cover a surgical device such as the surgical device 200. The surgical device may be, for example, a robotic arm such as the robotic arm 202, 316. The robotic arm may comprise a receiver such as the receiver 204 configured to receive a connector such as the connector 110 disposed on a second side of the flexible sheet. The robotic arm may also comprise a plurality of faces such as the plurality of faces 206. The marker may be positioned on a corresponding face so that the marker (and thus, the corresponding face) may be tracking by, for example, a navigation system such as the navigation system 318.

The information about the marker may be used to identify the corresponding face of the robotic arm. The information may be received from, for example, a processor such as the processor 304. The processor may obtain the information by, for example, using image processing such as the image processing 320 to process image data received from an imaging device such as the imaging device 312. The information may comprise, for example, a pose of the marker, a pattern defined by the marker, a QR code, or a barcode. In some embodiments, the information may be used in step 412 to identify the corresponding face of the robotic arm.

The method 400 also comprises receiving a pose of the robotic arm (step 408). The pose of the robotic arm may be received from sensors such as the sensors 326. The sensors may enable the processor (or a processor of a robot such as the robot 314) to determine a precise pose in space of the robotic arm. The sensors may positioned adjacent to or integrated with the robotic arm. The one or more sensors may include a plurality of sensors and each sensor may be positioned at the same location or a different location as any other sensor.

It will be appreciated that in some embodiments the method 400 may not include the step 408.

The method 400 also comprises identifying a face of the robotic arm (step 412). Identifying the face of the robotic arm may comprise the processor using an identifying content such as the identifying 322. The identifying enables the processor to identify the face of the surgical device corresponding to the marker using, for example the information received in step 404 and/or the pose of the robotic arm received in step 408. The identifying may, for example, enable the processor to match a unique identifier such as a unique pattern formed by the marker (whether the unique pattern is, for example, a combination of stickers in a unique pattern, a barcode, or a QR code) with a corresponding face. The unique pattern and the corresponding face may be predetermined and stored in, for example, a memory such as the memory 306, a database such as the database 330, or a cloud such as the cloud 334 and may accessed by the processor when executing the identifying. In other embodiments, the identifying may, for example, enable the processor to use the pose of the robotic arm received in, for example, step 408 and the information about the marker to identify the face.

The method 400 also comprises tracking the face (step 416). Tracking the face may comprise the processor using a tracking such as the tracking 324 to track the face. The tracking 324 enables the processor to track the face 206 identified. The tracking may, for example, enable the processor to compare the identified face at a first time period and a second time period to determine if movement of the identified face has occurred. In other embodiments, the tracking may, for example, enable the processor to compare a pose of the identified face (whether determined from image processing performed in step 404, receiving a pose from the surgical device in step 408, or otherwise) at a first time period and a second time period to determine a change in the pose (and thus, movement of the identified face).

The method 400 also comprises calibrating the navigation system (step 420). Calibrating the navigation system may occur, for example, prior to or after sterilization of the flexible sheet. Calibrating the navigation system may comprise comparing the pose of the marker (obtained from, for example, step 404) to an expected pose of the marker. A difference in the expected pose of the marker and the actual pose of the marker may be inputted into the navigation system as an offset. The navigation system may automatically apply the offset to, for example, a coordinate system used by the navigation system. Such calibration may beneficially increase an accuracy of the navigation system.

It will be appreciated that in some embodiments the method 400 may not include the step 420.

The present disclosure encompasses embodiments of the method 400 that comprise more or fewer steps than those described above, and/or one or more steps that are different than the steps described above.

As noted above, the present disclosure encompasses methods with fewer than all of the steps identified in FIG. 4 (and the corresponding description of the method 400), as well as methods that include additional steps beyond those identified in FIG. 4 (and the corresponding description of the method 400). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for covering and tracking a surgical device comprising:
a cover assembly comprising:
a flexible sheet configured to cover a surgical device;
at least one marker disposed on a first side of the flexible sheet;
a connector disposed on a second side of the flexible sheet, the second side opposite the first side; and
a receiver attached to a surgical device and configured to receive the connector, the receiver configured to align and hold the flexible sheet to the surgical device.

2. The system of claim 1, wherein the at least one marker is preconfigured on the flexible sheet prior to installation of the flexible sheet on the surgical device.

3. The system of claim 1, wherein at least one of the receiver or the connector comprises a magnet and at least another one of the receiver or the connector comprises steel.

4. The system of claim 1, wherein the surgical device comprises a robotic arm and the at least one marker corresponds to a face of a plurality of faces of the robotic arm.

5. The system of claim 4, further comprising:
a processor; and
a memory storing data for processing by the processor, the data, when processed, causing the processor to:
receive information about the at least one marker; and
identify a face of the robotic arm based on the information.

6. The system of claim 5, wherein the memory store further data for processing by the processor that, when processed, causes the processor to:
track the face.

7. The system of claim 5, wherein the information comprises a pattern formed by the at least one marker, the pattern corresponding to the face of the robotic arm.

8. The system of claim 5, wherein the information comprises a pose of the at least one marker, and wherein the memory store further data for processing by the processor that, when processed, causes the processor to:
receive a pose of the robotic arm, and
wherein identifying the face of the robotic arm is based on the pose of the robotic arm and the pose of the at least one marker.

9. The system of claim 1, wherein the receiver and the connector form a kinematic coupling.

10. The system of claim 1, wherein the at least one marker comprises at least one of a reflective sticker, a barcode, or a QR code.

11. The system of claim 1, wherein the flexible sheet forms a sterile barrier between the surgical device and a sterile environment.

12. The system of claim 1, wherein the receiver is at least one of integrated with the surgical device or attached to the surgical device.

13. A system for covering and tracking a surgical device comprising:
a flexible sheet configured to cover a surgical device;
at least one marker disposed on a first side of the flexible sheet;
a connector disposed on a second side of the flexible sheet, the second side opposite the first side;
a robotic arm having a receiver configured to receive the connector, the receiver configured to align and hold the flexible sheet to the surgical device;
a processor; and
a memory storing data for processing by the processor, the data, when processed, causing the processor to:
receive information about the at least one marker;
identify a face of the robotic arm based on the information; and
track the face.

14. The system of claim 13, wherein the information comprises a pose of the at least one marker, and wherein the memory store further data for processing by the processor that, when processed, causes the processor to:
receive a pose of the robotic arm, and
wherein identifying the face of the robotic arm is based on the pose of the robotic arm and the pose of the at least one marker.

15. The system of claim 13, wherein the receiver and the connector form a kinematic coupling.

16. The system of claim 13, wherein the information comprises a pattern formed by the at least one marker, the pattern corresponding to the face of the robotic arm.

17. The system of claim 13, wherein the at least one marker is preconfigured on the flexible sheet prior to installation of the flexible sheet on the surgical device.

18. The system of claim 13, wherein at least one of the receiver or the connector comprises a magnet and at least another one of the receiver or the connector comprises steel.

19. The system of claim 13, wherein the at least one marker comprises at least one of a reflective sticker, a barcode, or a QR code.

20. A device for covering and tracking a surgical device comprising:
a processor; and
a memory storing data for processing by the processor, the data, when processed, causing the processor to:
receive information about at least one marker disposed on a first side of a flexible sheet, the flexible sheet configured to cover and couple to a robotic arm via a connector disposed on a second side of the flexible sheet and a receiver disposed on the robotic arm;
identify a face of a robotic arm based on the information; and
track the face.
